(19) United States
(12) Patent Application Publication
(10) Pub. No.: US 2023/0338259 A1
(43) Pub. Date: Oct. 26, 2023

(54) HAIR LOTION

(71) Applicant: DERMACISEN, S.A., Ciudad de Panama (PA)

(72) Inventor: Jose Luis Cisneros Vela, Barcelona (ES)

(21) Appl. No.: 17/727,442

(22) Filed: Apr. 22, 2022

(51) Int. Cl. A61K 8/49; A61K 8/34; A61K 8/86; A61K 8/39; A61K 8/9794; A61K 8/44; A61K 8/9789; A61K 8/27; A61K 8/23; A61Q 5/12; A61Q 19/00; A61Q 7/00

(52) U.S. Cl. CPC A61K 8/4926; A61K 8/345; A61K 8/86; A61K 8/39; A61K 8/4953; A61K 8/9794; A61K 8/44; A61K 8/9789; A61K 8/27; A61K 8/23; A61K 8/4946; A61K 8/498; A61Q 5/12; A61Q 19/00; A61Q 7/00

(57) ABSTRACT

The present invention relates to a hair lotion for treating alopecia and for hair care, the composition thereof comprising the following products and percentages by weight, with a margin of ±5% with respect to the indicated values: solvent (87.634%), moisturizer (6.700%), emulsifier (1.450%), buffer (0.700%), preservative (1.292%), skin conditioner (1.213%), skin protectant (0.030%), chelating agent (0.350%), straightening agent (0.200%), anti-static agent (0.200%), tonic (0.150%), hair conditioner (0.080%), and antioxidant (0.001%).

### TECHNICAL FIELD

The present invention relates to a hair lotion applicable in the sector dedicated to the manufacture of hair care products and the purpose of which is to strengthen hair by increasing its density and preventing hair loss.

### PRIOR ART

Over the course of the 21^{st} century, medicine and well-being have been consolidated, and a cult to personal image, particularly in relation to hair care, caring for and treating alopecia, or hair loss, has emerged.

US patent 2020383916A1 describes topical formulations for treating dermatological disorders including male pattern baldness.

This document mentions that pattern hair loss in both men and women can be androgenic, where it is generally believed that male sex hormones play a major role in the health of hair follicles. Said hair follicles contain androgen receptors that bind with androgens, and the resulting complexes could cause miniaturization of hair follicles and reduce nutrients being supplied to hair shafts.

Said prior art document also refers to the use of finasteride for the treatment of hair loss in men, and the negative side effects detected in studies of finasteride, such as sexual dysfunction, where the use thereof is associated with a low libido, erectile dysfunction, decreased arousal, and problems with orgasm.

The technical problem considered is the development of a hair lotion for treating baldness which includes a series of active ingredients, for acting together with the inhibitors of 5-α reductase, the enzyme that transforms testosterone into dihydrotestosterone which is responsible for baldness.

### DISCLOSURE OF THE INVENTION

The composition of the hair lotion object of this invention includes products consisting of solvents, moisturizers, emulsifiers, buffer, preservatives, skin conditioners, skin protectants, chelating agents, straightening agents, anti-static agents, tonics, hair conditioners, and antioxidants.

The products indicated above have the percentages by weight indicated below, with a margin of ±5% with respect to the indicated values; said products include at least one of the components indicated below or a combination thereof:
Solvents (87.634%): aqua, alcohol denat, propylene glycol.
Moisturizers (6.700%): glycerin, ethoxydiglycol.
Emulsifiers (1.450%): polysorbate 80, laureth-9.
Buffer (0.700%): azelaic acid, sodium hydroxide, taurine.
Preservatives (1.292%): benzyl alcohol, phenoxyethanol, potassium sorbate, sodium benzoate.
Skin conditioners (1.213%): diaminopyrimidine oxide, caffeine, Serenoa serrulata fruit extract, arginine, Urtica dioica leaf extract, zinc gluconate, Rosmarinus officinalis leaf extract, Salvia officinalis leaf extract.
Skin protectants (0.030%): Panax ginseng root extract, Vitis vinifera seed extract.
Chelating agents (0.350%): citric acid, disodium EDTA.
Straightening agents (0.200%): niacinamide.
Anti-static agents: (0.200%): pyridoxine HCL.
Tonics (0.150%): Salvia officinalis oil.
Hair conditioners (0.080%): biotin, sulfur, Zea mays kernel extract.
Antioxidants (0.001%): tocopherol

The indicated components, in addition to the previously specified functions, have properties which make them particularly indicated for treating androgenetic alopecia or baldness, in both men and women, where the treatment with the hair lotion is based on five basic parameters:
1) Regenerator of the structural components of the hair follicle and shaft to maintain optimal hair quality.
2) Anti-seborrheic to reduce the degree of oily scalp.
3) Antioxidant with an anti-aging effect.
4) Inhibitor of the 5-α reductase enzyme.
5) Peripheral vasodilator for better nutrition and vascular irrigation of the hair.

### DETAILED DISCLOSURE OF EMBODIMENTS OF THE INVENTION

In a specific exemplary embodiment, the hair lotion object of the invention comprises the components and percentages by weight described below:

Solvents:
80.134% Aqua.
5.000% Alcohol denat.
2.500% Propylene glycol.

Moisturizers:
4.200% glycerin.
2.500% ethoxydiglycol.

Emulsifiers:
0.950% polysorbate 80.
0.500% laureth-9.

Buffer:
0.500% azelaic acid.
0.100% sodium hydroxide.
0.100% taurine.

Preservatives:
0.500% benzyl alcohol.
0.500% phenoxyethanol.
0.271% potassium sorbate.
0.021% sodium benzoate.

Skin Conditioners:
0.500% diaminopyrimidine oxide.
0.250% caffeine.
0.125% Serenoa serrulata fruit extract.
0.100% arginine.
0.010% Urtica dioica leaf extract.
0.200% zinc gluconate.
0.015% Rosmarinus officinalis leaf extract.
0.013% Salvia officinalis leaf extract.

Skin Protectants:
0.015% Panax ginseng root extract.
0.015% Vitis vinifera seed extract.

Chelating Agents:
0.250% citric acid.
0.100% disodium EDTA.

Straightening Agents:
0.200% niacinamide.

Anti-Static Agents:
0.200% pyridoxine HCL.

Tonics:
0.150% Salvia officinalis oil.

Hair Conditioners:
0.020% biotin.
0.050% sulfur.
0.010% Zea mays kernel extract.

Antioxidants:
0.001% tocopherol

In addition to the indicated properties, a significant part of the components has characteristics which are beneficial for treating androgenetic alopecia or baldness and which have a significant effect on the five basic parameters of the treatment with hair lotion indicated above.

Namely:
Caffeine, Urtica dioica leaf extract, Panax ginseng root extract, citric acid, pyridoxine HCL, Salvia officinalis oil, and biotin have a regenerative effect on the structural components of the hair follicle and shaft, which allows optimal quality to be maintained.
Arginine, zinc gluconate, niacinamide, and sulfur have anti-seborrheic properties which allow the degree of oily scalp to be reduced.
Salvia Officinalis seed extract and tocopherol are antioxidants that have an anti-aging effect on the skin.
Serenoa serrulata fruit extract is an inhibitor of the 5-α reductase enzyme, which is the main cause of baldness.
Rosmarinus officinalis leaf extract, Salvia officinalis leaf extract, and Zea mays kernel extract are peripheral vasodilators which facilitate vascular irrigation to nourish the hair.

The components included in this hair lotion act simultaneously on different factors causing alopecia, creating a synergy which provides effective treatment against alopecia or baldness.

Having sufficiently described the nature of the invention as well as a preferred embodiment thereof, it is hereby stated for all relevant purposes that the materials, shape, size, and arrangement of the described elements may be modified, provided that this does not entail an alteration of the essential features of the invention claimed below.

1. A hair lotion, suitable for treating alopecia and for hair care, the composition thereof comprising products consisting of solvents, moisturizers, emulsifiers, buffer, preservatives, skin conditioners, skin protectants, chelating agents, straightening agents, anti-static agents, tonics, hair conditioners, and antioxidants.

2. The hair lotion according to claim 1, wherein the products have the following percentages by weight, with a margin of ±5% with respect to the indicated values:
Solvents (87.634%);
Moisturizers (6.700%);
Emulsifiers (1.450%);
Buffer (0.700%);
Preservatives (1.292%);
Skin conditioners (1.213%);
Skin protectants (0.030%);
Chelating agents (0.350%);
Straightening agents (0.200%);
Anti-static agents (0.200%);
Tonics (0.150%);
Hair conditioners (0.080%);
Antioxidants (0.001%).

3. The hair lotion according to claim 1, wherein the products include at least one of the following components:
Solvents: aqua, alcohol denat, propylene glycol;
Moisturizers: glycerin, ethoxydiglycol;
Emulsifiers: polysorbate 80, laureth-9;
Buffer: azelaic acid, sodium hydroxide, taurine;
Preservatives: benzyl alcohol, phenoxyethanol, potassium sorbate, sodium benzoate;
Skin conditioners: diaminopyrimidine oxide, caffeine, Serenoa serrulata fruit extract, arginine, Urtica dioica leaf extract, zinc gluconate, Rosmarinus officinalis leaf extract, Salvia officinalis leaf extract;
Skin protectants: Panax ginseng root extract, Vitis vinifera seed extract;
Chelating agents: citric acid, disodium EDTA;
Straightening agents: niacinamide;
Anti-static agents: pyridoxine HCL;
Tonics: Salvia officinalis oil;
Hair conditioners: biotin, sulfur, Zea mays kernel extract;
Antioxidants: tocopherol.

4. The hair lotion according to claim 4, comprising the following percentages by weight of each component:
Solvents:
80.134% Aqua,
5.000% Alcohol denat,
2.500% Propylene glycol;
Moisturizers:
4.200% glycerin,
2.500% ethoxydiglycol;
Emulsifiers:
0.950% polysorbate 80,
0.500% laureth-9;
Buffer:
0.500% azelaic acid,
0.100% sodium hydroxide,
0.100% taurine;
Preservatives:
0.500% benzyl alcohol,
0.500% phenoxyethanol,
0.271% potassium sorbate,
0.021% sodium benzoate;
Skin conditioners:
0.500% diaminopyrimidine oxide,
0.250% caffeine,
0.125% Serenoa serrulata fruit extract,
0.100% arginine,
0.010% Urtica dioica leaf extract,
0.200% zinc gluconate,
0.015% Rosmarinus officinalis leaf extract,
0.013% Salvia officinalis leaf extract;
Skin protectants:
0.015% Panax ginseng root extract,
0.015% Vitis vinifera seed extract;
Chelating agents:
0.250% citric acid,
0.100% disodium EDTA;
Straightening agent:
0.200% niacinamide;
Anti-static agent:
0.200% pyridoxine HCL;
Tonic:
0.150% Salvia officinalis oil;
Hair conditioners:
0.020% biotin,
0.050% sulfur,
0.010% Zea mays kernel extract; and
Antioxidants:
0.001% tocopherol.
